(19) United States
(12) Patent Application Publication
(10) Pub. No.: US 2009/0036410 A1
(43) Pub. Date: Feb. 5, 2009

(54) Structured Phospholipids

(75) Inventors: Laurence S. Harbige, London (GB); Michael J. Leach, London (GB); Mohammed Sharief, London (GB); Paul Barraclough, Kent (GB)

Correspondence Address: NIXON & VANDERHYE, PC, 901 NORTH GLEBE ROAD, 11TH FLOOR, ARLINGTON, VA, 22203, US

(73) Assignee: BTG INTERNATIONAL LIMITED, London (GB)

(21) Appl. No.: 11/791,606

(22) PCT Filed: Nov. 25, 2005
(86) PCT No.: PCT/GB05/04516

§ 371 Date: Feb. 13, 2008

(30) Foreign Application Priority Data
Nov. 25, 2004 (GB) 0425932.1

(51) Int. Cl. A61K 31/661; A61P 37/00; A61P 35/00; C07F 9/09

(52) U.S. Cl. 514/114; 554/82

(57) ABSTRACT

A method of treating a patient in need of therapy for a disease in which cyokines have become dysregulated, or are otherwise capable of modulation to provide therapeutic benefit, is provided comprising administering to that patient a therapeutically effective dose of a phospholipid comprising a phosphatidyl group esterifed with one or more fatty acyl groups, characterised in that the phospholipid has at least one fatty acyl group at the sn-1 and/or sn-2 position of the phosphatidyl group, the fatty acyl group being selected from the group consisting of γ-linolenoyl, dihomo-γ-linolenoyl acid and arachidonoyl.

The present invention relates to a method for modulating cytokine levels in subjects sufferring from diseases in which these have become dysregulated or are otherwise capable of modulation to provide thearpeutic benefit. Particularly is provided a method for modulating transforming growth factor β (TGF-β), particularly TGF-β1, but also cytokines TNF-α and IL-1β, still more preferably for maintaining and/or restoring cytokine balance where imbalance is found in diseases of the immune system and in neurodegeneration. Such diseases include multiple sclerosis and various autoimmune states.

More particularly the present invention provides treatment for neurodegenerative conditions, particularly those such as demyelinating diseases, such as multiple sclerosis, Alzheimer's and Parkinson's diseases and the degenerative sequelae associated with head trauma, stroke and intracranial bleeds, whereby neuronal function may be improved or restored from an impaired condition, eg. by remyelination.

Further provided are novel use of known and novel compounds comprising unsaturated fatty acid moieties for the manufacture of medicaments capable of effectively treating such conditions, more particularly being capable of achieving previously unattained levels of success with regard to maintenance and recovery of neurological function.

It is well reported in the literature that essential fatty acids (EFAs) of the n-3 and n-6 unsaturation pattern have beneficial effect in a wide variety of human physiological disorders, including autoimmune diasese (WO 02/02105). Harbige (1998) Proc. Nut. Soc. 57, 555-562 reviewed the supplementation of diet with n-3 and n-6 acids in autoimmune disease states, and particularly noted evidence of benefit of γ-linolenic (GLA) and/or linoleic acid (LA) rich oils. Harbige (2003) Lipids Vol 38, no 4 discusses broader implications for the immune system and the mechanisms whereby high LA supplementation might cause production of pro-inflammatory states.

The inventor's copending unpublished patent application PCT/GB2004/002089 and PCT/GB2004/003524, incorporated herein by reference, relate to the use of synthetic, plant and fungal oils for the treatment of neurodegenerative diseases, particularly multiple sclerosis, stroke, head trauma, Alzheimer's and Parkinsons's disease. PCT/GB2004/002089 relates to oils characterised by having at high percentages of the essential fatty acid γ-linolenic acid (GLA) at the sn-2 position of their lipids, typically being over 40% of the sn-2 fatty acid total of the oil. PCT/GB2004/003524 relates to structured lipids having an sn-2 fatty acid residue selected from γ-linolenic acid (GLA), dihomo-γ-linolenic acid (DHGLA) and arachidonic acid (AA).

These applications report remakable levels of success in treating animal model CREAE and human relapse remitting multiple sclerosis. When triglyceride oils containing suitable levels of these fatty acids in the sn-2 position are administered to patients over a period of several months the inventors have determined an associated therapeutic increase in TGF-β1 and decrease in TNF-α and IL-1β, as measured as spontaneously released from Peripheral Blood Mononuclear Cells (PBMC) isolated from a patient's blood.

The present inventors unpublished work, described in the aforesaid PCT applications, has indicated that the position of the γ-linolenic acid, dihomo-γ-linolenic acid and arachidonic acid in a glyceride is of great significance in determining its activity in modulating cyctokines and in correcting metabolic defect in multiple sclerosis and other demyelinating disease. Whereas sn-1 and sn-3 position fatty acids appear to have very little therapeutic significance, the sn-2 position is critical to the activity of the triglyceride.

The prior art does not appear to recognise this crucial fact, with consequences of failure in all previous studies. Table 3 of EP 0520624 (Efamol Holdings) compares the triglyceride content of Evening Primrose and Borage Oils, the former being taught to be more therapeutically effective than the latter for a variety of GLA responsive disorders. This document indicates Borage oil to have at least twenty seven different trigyceride components, only 20% by weight of which of which have sn-2 GLA. Page 3, lines 40-42 notes that biological testing has shown that equal amounts of GLA may indeed have very different effects when that GLA is supplied as different oil sources. Crucially, it then directs the reader to one particular fraction present in Evening Primrose Oil (EPO), but not Borage Oil, as being responsible for the former's superior effect in raising PGE₁ (see EP 0520624 Chart page 4 and Table 2) and thus the anti-inflammatory effect: that fraction being identified as di-linoeoyl-mono-gamma-linolenyl-glycerol (DLMG) which it states to be 18 to 19% of the total triglyceride in EPO. Crictically, page 6 clearly teaches that the position of the GLA, in sn-1, 2 or 3, is not important to this effect.

Dines et al (1994) Proceedings of the Physiological Society, Aberdeen Meeting 14-16 September 1994 report on studies of treatment of diabetic neuropathy neuronal damage with γ-linolenic acid containing oils of the type advocated by EP 0520624 and again note that Borage Oil was not very effective in treating this neurodegeneration whereas Evening primrose oil was. The paper concludes that Borage Oil contains other constituents that interfere with GLA activity.

Bates et al noted that lipid oils comprising a mixture of linoleic acid and γ-linolenic acid residues had been suggested back in 1957 to be possibly more efficacious in treating inflammation and autoimmune diseases, but found that at 3 g oil per day (Naudicelle Evening Primrose oil 7:1 LA:GLA), patients who had relapses became more ill on the trial oil than on the control.

Although the aetiology of multiple sclerosis (MS) remains unknown, studies have shown that MS patients have higher than normal neuro-antigen autoreactive T-cells levels. These T-cells react inter alia to myelin basic protein (MBP) and myelin oligodendrocyte glycoprotein (MOG) and are in an increased state of activation compared with healthy controls. The actual processes of axonal damage e.g. chronic inflammation, demyelination and astrogliosis in MS is complex, but white matter inflammation and demyelination are considered to determine disease severity, whilst recent studies suggested that axonal damage in MS begins in the early stages of the disease and contributes to disability (De Stefano et al, 2001).

Experimental autoimmune encephalomyelitis (EAE) is the most frequently used animal model for immune mediated effects of MS. Studies in the guinea-pig have shown that linoleic acid partially suppresses the incidence and severity of EAE (Meade et al (1978)). (Harbige et al (1995), 1997b) demonstrated disease modifying effects of linoleic acid and γ-linolenic acid on clinical and histopathological manifestations of EAE. Depending on dose, γ-linolenic acid was fully protective in acute rat EAE whereas linoleic acid had dose-dependent action on the clinical severity but did not abolish it.

Despite these experimental findings, it is recognised that the human disease, multiple sclerosis, is highly complex and can be conversely exacerbated and ameliorated by the activity of T-cells and other immune response factors. It is thought that the n-6 fatty acids promote autoimmune and inflammatory disease based upon results obtained with linoleic acid only. TGF-β1 and PGE₂ production has been shown to be increased non-specifically in γ-linolenic acid fed mice ex vivo.

Cytokines are implicated in the pathogenesis of MS, with many studies showing an increase in myelinotoxic inflammatory cytokines (TNF-α, IL-1β and IFN-γ) coinciding with the relapse phase of the disease. TGF-β1 has been reported to protect in acute and relapsing EAE ((Racke et al (1993); Santambrogio et al (1993)), and PG inhibitors such as indomethacin augment, and thus worsen, the disease (Ovadia & Paterson (1982)).

Conversely, levels of the anti-inflammatory and immunosuppressive cytokine transforming growth factor-betal (TGF-β1) appear to be reduced during a phase of relapse and increase as the patient enters remission. Thus the balance between biologically active TGF-β1 and the pro-inflammatory TNF-α, IL-1β and IFN-γ appears to be dysregulated during MS relapse-remission.

During natural recovery phase from EAE, TGF-β1-secreting T-cells inhibit EAE effector cells, TGF-β1 is expressed in the CNS and, in oral-tolerance-induced protection in EAE, TGF-β and PGE₂ are expressed in the brain (Karpus & Swanborg (1991); Khoury et al (1992)). Harbige ((1998) concluded that dietary γ-linolenic acid effects on EAE are mediated through Th₃-like mechanisms involving TGF-β1 and possibly through superoxide dismutase antioxidant activity.

In spite of the use of Borage oil and other γ-linolenic acid/linoleic acid containing oils such as Evening Primrose oil by multiple sclerosis sufferers over the past 30 years or so, the vast majority of patients fail to recover from the disease, showing no significant improvement, with the underlying disease continuing to progress to death.

It has been suggested to use, inter alia, γ-linolenic acid and linoleic acid rich Borage oil as a means to provide immuno-suppression in multiple sclerosis (U.S. Pat. No. 4,058,594). Critially, the dose suggested is 2.4 grams of oil per day and no actual evidence of efficacy is provided. This is much lower than the low 5 gram/day dose that the present inventors have now found to be ineffective in vivo in man as reported in PCT/GB04/002089, indeed the inventors have found that doses as high as 10 gram/day have been found to be ineffective in some patients.

Other more dramatic immunosuppressant treatments, including T cell depleters and modulators such as cyclophosphamide, are also shown to be effective in the EAE model, but where these are employed in the human multiple sclerosis disease symptoms improve, but the underlying disease continues to progress. This is probably because T-cells indeed produce beneficial cytokines, such as TGF-β1, as well as deleterious ones in man.

David Baker of Institute of Neurology, UK summed up the disparity between what is effective in the EAE and in MS with a paper entitled ‘Everything stops EAE, nothing stops MS’ at the 10^{th} May 2004 UK MS Frontiers meeting of the UK MS Society. It is clear that immunosuppression alone cannot cure MS. This is almost certainly due to a fundamental underlying metabolic disorder in MS patients (Hollifield et al (2003) Autoimmunity, Vol 36, p 133-141), in addition to the autoimmune disease, that leads to membrane abnormality, cytokine dysregulation and subsequent immune attack and lesioning. Although patients go into remission in relapse-remitting disease, the underlying demyelination proceeds.

The ‘gold standard’ treatment for MS remains interferon, such as with β-Avonex ®, Rebif ® er interferon preparations. This gold standard treatment only addresses needs of some, eg 30%, of the patients and even in these symptom improvement is restricted to reduced severity of relapses. Whilst symptoms may be reduced in a proportion of patients, the disease tends to progress to further disability and death due to underlying degeneration.

The copending PCT/GB2004/002089 and PCT/GB2004/003524 show that adminstration of a suitably high level of γolenic acid, dihomo-γolenic acid and/or arachidonic acid as lipid sn-2 position fatty acid residue, is capable of achieving the immunoregulation and metabolic defect correction that is required to successfully arrest the otherwise inevitable decline seen in multiple sclerosis patients.

However, the inventors are aware that it is desirable to achieve as much efficiency as possible when adminstering these fatty acids if a patient that is taking such doses long term is to been kept from entering a pro-inflammatory, and thus disease worsening state. Any γolenic acid, dihomo-γolenic acid and arachidonic acid that dose not enter the membrane can end up as free fatty acid overflowing into other pools, eg. resulting in Th-2 like humoral responses and increasing neutrophil mediated inflammation. To this end the inventors have now developed their unpublished invention further by preparing and testing structured phospholipids that are still more efficacious at directing these key fatty acid to the cell membranes but not elsewhere.

Two pools of polyunsaturated fatty acids (PUFA) are thought to exist for eicosanoid (e.g. prostaglandin) biosynthesis: a metabolic pool and a membrane-bound pool. There is little doubt that free arachidonic acid is converted into prostoglandins but there is little, if any, free arachidonic acid available under “normal” conditions as most is bound up in phosphoglycerides. Basal prostaglandin synthesis obtains its fatty acid precursors from a metabolic pool which starts from linoleic acid (18:2n-6) and is consequently linked to dietary intake (linoleic acid is quantitatively the major PUFA found in the diet) and that are found in the neutral lipid pool e.g. triacylglcerols and free fatty acids.

The substrates for this metabolic pool would be derived from dietary fats and from their mobilization in adipose tissue. Normally the longer-chain highly unsaturated fatty acid derivatives (e.g. γlinolenic acid, dihomo-γlinolenic acid and/or arachidonic acid) are at low levels in the normal diet and are differentially incorporated directly into phosphoglycerides (phospholipids pool). The phosphoglyceride (phospholipids) pool becomes active under conditions of trauma (e.g. inflammation) rather than the metabolic pathway. This is of course the classic phospholipase, free arachidonic acid, cyclooxygenase, lipoxygenase pathway.

Thus when a patient is fed high levels of these three n-6 fatty acids, not normally seen the diet, there is optimal incorporation of these fatty acids into phosphoglycerides, but once that level has been reached there is “overload” and these biologically highly active species will spill into the metabolic pool and be oxidised e.g. to the highly vasoactive prostaglandins. The optimal incorporation may vary under different disease conditions

In consequence, for triglycerides, there can be only a limited amount of sn-1 and sn-3 γ-linolenic acid, dihomo-γ-linolenic acid and/or arachidonic acid that can be tolerated as once these are released by lipases in the gut or in the mucosal cell (enterocyte) they are “free”. Although some re-esterification into triglyceride takes place in the mucosal cell, the bulk will enter the metabolic pool and alter the normal homeostatic processes, controlled by e.g. prostaglandins, such as vascular physiology.

It is known that atherogenic potential is associated with saturated and monounsaturated C20 to C24 fatty acids (particularly the very long chain C22 and their monenes), but not with C2-8, C10, C12, C14, C16 and C18 fatty acids. The latter are therefore preferred fatty acids for use in the structured lipids of the copending and present patent applications in positions where fatty acids will be lost to the metabolic pool.

The inventors have determined that the level of sn-1 linoleic acid may account for the lower potency of borage oil vs fungal oil shown in prior art EAE studies through competition and conversion to 20:2n-6 which will compete in membrane phospholipid sn-1 and sn-2 with γ-linolenic acid, dihomo-γ-linolenic acid and/or arachidonic acid. A mucosal cell 1,2-diglyceride pathway exists that gives rise to glycerophospholipids which may also be important regards competition between linoleic acid and these acids and/or linoleic acid's effects on micelle solubility.

Fat digestion in the small intestine (duodenum) involves pancreatic lipase which hydrolyzes the sn-1 and sn-3 positions of triacylglycerols after these are emulsified with bile salts (triacylglycerols or triglycerides are the major fat in diet, with much smaller amounts of phospholipids being present). The products of this digestion are free fatty acids and sn-2 monoglycerides. Micellar formation solubilizes the monoglycerides and fatty acids. This process appears to be affected by the presence of phospholipids and monoglycerides, furthermore mixed micelles e.g. containing oleic and linoleic acid (monoolein and monolinolein) appear to be better absorbed and improve the absorption of other fatty acids.

Monoglycerides, dietary cholesterol and fatty acids from the micelles enter the mucosal cells by passive diffusion. Fatty acids of C10-12 carbon or less pass from the mucosal cells directly into the portal blood, where they are transported as free (unesterified) fatty acids to the liver. This is the basis for the clinical use of medium chain triglycerides (MCTs) in bums, surgery, trauma etc. Fatty acids containing more than 10-12 carbon atoms are re-esterified to triglycerides in the mucosal cells (see FIG. 1). In the mucosal cells, most of the triglyceride is formed by the acylation of the absorbed 2-monoglycerides at the smooth endoplasmic reticulum. The new triglyceride, which retains sn-2 γ-linolenic monoglycerides, is transported (after packaging with a protein [B48,CII,AI] component within the mucosal cell Golgi) in chylomicrons to the central lacteal of the villus and carried in the lymphatic system (FIG. 1). Lymph vessels course between the layers of the mesentery to the pre-aortic lymph glands and empty into the thoracic duct to the systemic circulation.

Circulating dietary triglycerides, as part of chylomicons, are transported to the liver and also removed from the blood, as they are in the lymphatics, by lipoprotein lipase on the luminal side of capillary beds in e.g. muscle, heart, adipose tissue. Lipoprotein lipase acts on the sn-1 and sn-3 fatty acids supplying e.g. adipocytes with fatty acids which then re-esterify the fatty acids into triglycerides (fat deposition). Release by adipose tissue surrounding lymph nodes (this adipose tissue preferentially incorporates PUFA) supplies the node with fatty acids for membrane synthesis (PUFA) and energy (saturates).

Thus dividing/proliferating lymphocytes (greatly increased under EAE, CREAE and other autoimmune conditions) incorporate fatty acids, allowing for triglyceride incorporation directly into lymphocyte membranes. γ-linolenic acid will require conversion to dihomo-γ-linolenic acid and arachidonic acid and it is then there will be an impact on the cytokine production pattern of the T-lymphocytes, under activation conditions, to a localised T cell TGF-β1 (T regulatory cell) dominated response rather than a γ-IFN dominated T cell response (note these are the effector T cells that mediate EAE). PGE₂ production by regulatory macrophages/monocytes is also potentially important.

In contrast to the triglycerides of copending PCT/GB2004/003524, it appears that dietary phospholipids are acted on by a pancreatic phospholipase A2 in the intestine releasing the sn-2 fatty acid present (usually an unsaturated fatty acid such as linoleic or arachidonic acid) and forming a lysophosphatidyl moiety (sn-2 lyso-phospholipid). The unsaturated fatty acids released are absorbed as free fatty acids and reincorporated into glycerolipids which are made into new phospholipids (FIG. 1) in the rough endoplasmic reticulum.

Thus during fat absorption chylomicron phospholipids are derived from reacylation of the absorbed sn-2 lysophosphatidyl compound (eg phosphotidylcholine), increased de novo synthesis and mucosal phospholipid pools. It is also thought that there is preferentially reacylation, although the specificity of the intestinal 1-lyso-PC-acyl-CoA-acyltransferase has not been fully studied, of γ-linolenic acid, dihomo-γ-linolenic acid, arachidonic acid and to a lesser extent linoleic acid into the 1-lyso-PC (sn-1 of PC). Thus the theory is that the sn-1 in eg. phosphatidylcholine, should remain relatively intact which would target the sn-1 fatty acid to the membrane pool.

It appears from the limited published data and the inventors own observations that the preferential transport of chylomicron phospholipids, combined with a positional specificity of lipoprotein lipase and hepatic and lymphoid tissue lipases for sn-1 of phospholipids such the phosphatidyl esters PC (phosphatidylcholine) and PE (phosphatidylethanolamine), is important. It provides a physiologically important transport system for high amounts of the biologically potent longer chain n-6 fatty acids which can be distributed to various organs/tissues without any risk of uncontrolled physiological effects caused by intravascular release of these fatty acids, particularly arachidonic acid, in their free unesterified form.

It is known that preferential incorporation of free arachidonic acid (orally dosed) into the chyle phospholipids occurs, but on giving high doses, arachidonic acid-rich triglycerides are also observed, indicating overflow into the triglyceride pool. It should be appreciated that chylomicrons transporting lipids e.g. phospholipid are providing lipids for the growth of body tissues/cells/membranes and can be directly incorporated into membranes.

In contrast the triglyceride fatty acids are stored in adipose tissue and released, in the case of lymphoid tissues to provide additional fatty acids, both saturated and unsaturated, for membrane incorporation in actively proliferating lymphocytes. It should be noted that lymphocytes preferentially use glutamine and fatty acids as their metabolic fuel rather than glucose; there maybe however direct triglyceride incorporation.

The present invention provides new phospholipids, particularly but not exclusively 3-sn-phophatidyl esters, and identifies known phospholipids that will deliver γ-linolenic acid, dihomo-γ-linolenic and arachidonic acid directly into the cell membrane with much reduced risk of free fatty acid release. Should γ-linolenic acid not undergo sufficient metabolic conversion by this route, perhaps due to individual patient idiosyncracy, the preformed dihomo-γ-linoleic acid or arachidonic acid phospholipids will be more active. In addition these enriched phospholipids have a potential role in membrane reparation, such as for neural membranes in multiple sclerosis, where underlying δ-6 desaturase activity is now thought to be deficient.

It is known from EP0609078 and U.S. Pat. No. 5,466,841 to prepare phospholipids including two different unsaturated fatty acids selected from the twelve n-6 and n-3 essential fatty acids (EFA), oleic acid and combinic acid. The use of such phospholipids is said to be for administration of a single component molecule that can provide supplementation for dietary essential fatty acid insufficiency. The implication of this teaching is that two essential fatty acids may be supplemented in one molecule, with no preference being given to the position of the EFA at sn-1 or sn-2 the phosphotidyl group being at sn-3). These patents/applications teach preparation and use of phosphatidyl-serine, phosphatidyl-choline, phosphatidyl-ethanoloamine and phosphatidyl-inositol derivatives of the EFAs.

U.S. Pat. No. 3,577,446 describes the synthesis of phosphatidylalkanolamines, particularly 1,2-di-(octadeca-9,12,15-trienoyl)-sn-glycero-3-phosphorylethanolamine useful as antihypertensive agents. This has two identical fatty acid residues attached to the sn-1 and sn-2 residues of a phosphatidylethanolamine group. JP 63-225387, JP 3-153628 and JP 61-129190 all describe phosphatidyl based infusions. EP0147741 describes 1,2-diacylglycero-3-phosphatidy-cholines as additives in tablets at levels of 100 mg per tablet.

In the present application the inventors describe the use of 1,2-diacyl-3-phosphatidyl esters of n-6 fatty acids for the treatment of diseases requiring modulation of dysregulated cyctokines, these cytokines being particularly TGF-β1, but also cytokines TNF-α and IL-1β.

Diseases that are treated are particularly neurodegenerative conditions, particularly those such as demyelinating diseases, such as multiple sclerosis, Alzheimer's and Parkinsons diseases and the degenerative sequelae associated with head trauma, stroke and intracranial bleeds, whereby neuronal function may be improved or restored from an impaired condition, eg. by remyeleination.

Particularly however the present invention relates to the treatment of multiple sclerosis, more prefereably where the patient has deficits in TGFβ1 release from PBMCs and/or a deficit in arachidonic acid levels in PBMCs. Most prefereably the disease treated is relapse remitting MS, secondary progressive MS or primary progressive MS.

A key advantage of the present invention comes with the realisation that the position of the essential fatty acyl groups γ-linolenoyl, dihomo-γ-linolenoyl and arachidonoyl in a lipid has significance for its therapeutic efficacy, as set out in the aforesaid PCT application and theorised above. This may be particularly serious where free fatty acid release results in arachidonic acid overdose, but may also be produced with overdose of the precursors γ-linolenic acid and dihomo-γ-linolenic acid. Paradoxically, previous treatment regimens using γ-linolenic acid rich oils, such as Evening Primrose Oil and lower sn-2 content Borage Oils, have provided too little sn-2-GLA to have any effect, as demonstrated by PCT/GB2004/002089's low dose (5 g/day).

In a first aspect the present invention provides a method of treating a patient in need of therapy for a diseases in which cyokines have become dysregulated, or are otherwise capable of modulation to provide therapeutic benefit, comprising administering to that patient a therapeutically effective dose of a phospholipid comprising a phosphatidyl group esterifed with one or more fatty acyl groups, characterised in that the phospholipid has at least one fatty acyl group at the sn-1 and/or sn-2 position of the phosphatidyl group, the fatty acyl group being selected from the group consisting of γ-linolenoyl, dihomo-γ-linolenoyl acid and arachidonoyl.

Preferably is provided a method for treating a patient in need of modulation of transforming growth factor β (TGF-β), particularly TGF-β1, but also cytokines TNF-α and IL-1β, still more preferably for maintaining and/or restoring cytokine balance where imbalance is found in diseases of the immune system and in neurodegeneration. Such diseases include multiple sclerosis and autoimmune disease states.

Particularly there is provided a method of treating a patient in need of therapy for a dysregulated or beneficially modulatable cytokine disease, particularly a neurodegenerative disease, comprising administering to that patient a therapeutically effective dose of a defined structure phospholipid comprising a phosphatidyl group esterifed with one or more fatty acyl groups, characterised in that the lipid has at least one fatty acyl group at the sn-1 and/or sn-2 position of the phosphatidyl group selected from the group consisting of γ-linolenoyl, dihomo-γ-linolenoyl acid and arachidonoyl.

More preferably the phospholipid has a fatty acyl group selected from the group consisting of γ-linolenoyl acid, dihomo-γ-linolenoyl acid and arachidonoyl at only one of the sn-1 or sn-2 positions of the phosphatidyl group, the other position being free hydroxyl or esterified with a C2 to C36 unsaturated, monounsaturated or polyunsaturated fatty acyl group.

Still more preferably the phospholipid has a an sn-1 position fatty acyl group selected from γ-linolenoyl, dihomo-γ-linolenoyl and arachidonoyl and an sn-2 position fatty acid selected from C2 to C36 unsaturated, monounsaturated or polyunsaturated fatty acyl other than n-6 acids.

Most preferably the other fatty acid is such that it is used in the metabolic pool, eg. being unsaturated or a metabolically acceptable acid such as oleic or palmitic acid.

The phospholipid phosphatidyl group is preferably selected from those found in mammalian, particularly human, cell membranes, more preferably is selected from the group consisting of phosphatidyl-choline, phosphatidyl-ethanolamine, phosphatidyl-serine, phosphatidyl- inositol, plasmalogens of the above e.g. lyso-phosphatidyl-choline, lyso-phosphatidyl-ethanolamine, lyso-phosphatidyl-inositol and lyso-phosphatidyl-glycerol.

Particularly advantageously treated neurodegenerative diseases are those involving demyelination. The present method specifically arrests underlying neurodegeneration and restores neuronal function. Particularly the method normalises membrane composition, in immune cells and neurones, and restores healthy PBMC spontaneously released TGF-β1/TNFα ratios and the ratios of TGF-β1 with other PBMC released cytokines.

Most advantageously the method arrests neurodegeneration in multiple sclerosis of all types but particularly relapsing remitting, primary and secondary progressive and other chronic progressive MS and the restoration, in part or completely, of neuronal function such as measured, eg. By MRI or CAT scan or by EDSS score. EDSS score preferably is improved by at least one point, more preferably at least 1.5 points and most preferably by at last 2 points over 18 months of daily treatment. Such method may also be used in treatment of cerebral impairment after stroke, head trauma and intracranial bleeding where there is infarct, eg. demyelination or neuronal damage. Further application is provided in treating other chronic demyelination such as in Alzheimer's and Parkinson's disease.

Preferably the the phospholipid is administered for a duration and at a dose sufficient to maintain or elevate TGF-β1 levels in the patient to therapeutic levels. By therapeutic levels is meant levels at least consistent with healthy subjects. Preferably the dose is such as to produce a TGF-β1/TNF-α ratio spontaneously released from peripheral blood mononuclear cells (PBMCs) isolated from blood of a patient, after 18 months of daily dosing, of 0.4 to 3.0, at least 0.5, more preferably at least 0.75 and most preferably at least 1. Preferably the dose is such as to produce a TGF-β1/IL-1β ratio in blood of a patient, after 18 months of daily dosing, of at least 0.5, more preferably at least 0.75 and most preferably at least 1. Preferably said levels are produced after 12 months and more preferably after 6 months.

Examples of healthy TGF-β1 are 80 pg/ml or more per 2×10⁶ cells spontaneously released from peripheral mononuclear blood cells isolated from the patient, more preferably above 100 pg/ml and most preferably above 140 pg/ml, still more preferably greater than 180 pg/ml. Methods for measuring this release are described in the Examples section herein.

Typically the amount of phospholipid administered daily will be between 0.5 and 30 grams, orally dosed, still more preferably between 0.5 and 20 grams and most preferably between 0.5 and 10 grams, typically 1 to 8 grams and most preferably between 1.2 and 3 grams.

Where the obligate (ie. the fatty acyl that is required to be present) sn-1 or sn-2 fatty acyl group is γ-linolenoyl, the dose may be toward the higher end of these ranges, particuarly where the other sn-1 or sn-2 group is relatively inert, eg. being metabolically utilised acids such as saturated fatty acids. Where the obligate sn-1 or sn-2 fatty acyl group is dihomo-γ-linolenoyl, the dose may be less, whilst where it is aracidonoyl, efficacy is higher, but dosing should be more cautious, due to possibilities of unwanted side effects at higher levels.

More preferably the method is characterised in that the phospholipid is a monoacyl or diacylphosphoglyceride, containing the at least one sn-1 or sn-2 γ-linolenoyl, dihomo-γ-linolenoyl or arachidonoyl group, of general Formula I below:

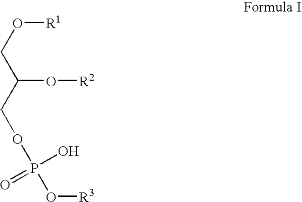

wherein R¹ and R² are independently selected from the group consisting of hydrogen, γ-linolenoyl, dihomo-γ-linolenoyl and arachidonoyl, mono-unsaturated C₃₋₃₆, linoleoyl or n-3 polyunsaturated acyl groups and optionally substituted C₂₋₃₆ saturated acyl, and

R³ is selected from moieties that are conjugated to phosphatidyl groups naturally occurring in mammalian membranes with the condition that one of R¹ and R² must be selected from γ-linolenoyl, dihomo-γ-linolenoyl and arachidonoyl.

For the purpose of the present invention the C₂₋₃₆ acyl groups comprise at least one carbonyl group on the end of a hydrocarbyl chain selected from alkyl and alkenyl chains, the carbonyl group being directly attached by its carbon to the oxygen of the glycerol residue shown in Formula I

Preferred acyl groups R¹ and R², when they are not γ-linolenoyl, dihomo-γ-linolenoyl and arachidonoyl, are saturated acyl of formula —CO—(CH₂)ₙ—CH₃, wherein n is an integer selected from 1 to 22, more preferably being 4 to 16, still more preferably being from 5 to 12, most preferably being from 6 to 10. Particularly preferred acyl groups are those of caprylic and capric acids, particularly being 1,3-dicaprylic or 1,3-dicapric glycerols having the γ-linolenoyl, dihomo-γ-linolenoyl or arachidonoyl group at the sn-1 or sn-2 position, most preferably the sn-1 position.

Preferred groups R³ are polar groups such as choline, ethanolamine, serine, inositol and glycerol. Other naturally occuring groups R³ will occur to those skilled in the art in the light of these but may be tissue specific, eg. specific to T-cells or nervous tissue. Other groups are preferably bipolar substituted C₁₋₁₀ alkyl or alkenyl groups substituted with eg amine, hydroxy or thio at one end and hydroxyl at the other such that there is formed a phosphotidate ester with the polar group.

Whilst most preferred groups R¹ to R² for inclusion in the compound of formula I in addition to one of the three obligatory (‘obligate’) n-6 acyl groups, are simple saturated fatty acyl or naturally occurring fatty acyl with structural or metabolic function, such as medium chain or long chain fatty acyl, there are other possibilities. Particularly preferred fatty acyls are those that are utilised primarily by the metabolism for producing energy. Other preferred acyls for sn-1 and sn-2 are selected from fatty acyls that are metabolised in the human to yield energy as opposed to a fatty acid that is primarily directed to the structural membrane pool: such preferred acids include oleic acid and palmitic acid.

Where used herein residue with respect to the phospholipid, in respect of acyl, particularly fatty acyl, groups means the moiety that remains after the fatty acid carboxyl group esterifies to one of the hydroxy groups of the glycerol molecule.

Where the other, non-obligate, sn-1 and sn-2 fatty acid chain (R¹ and R²) is unsaturated it may also be that of other essential fatty acids, such as the n-3 acids such as stearidonic acid, eicosapentanoic acid and docosahexanoic acid.

The non-obligate fatty acyl may be optionally substituted and these substitutions will preferably be by hydroxy, oxo, carboxyl, alkyl, alkenyl and alkoxy groups. Many naturally occurring substituted fatty acyls exist, eg. such as (R)-3-hydroxybutyrate and acetoacetate.

In a second aspect of the present invention there is provided the use of a compound of formula I, as defined above and with preferences of the method, for the manufacture of a medicament for the treatment of the diseases of the method of treatment.

In a third aspect of the invention there is provided a pharmaceutical composition for the treatment of a patient in need for modulation of dysregulated cytokines or cytokines which are otherwise capable of modulation to provide therapeutic benefit characterised in that it comprises a phospholipid comprising a phosphatidyl group esterifed with one or more fatty acyl groups, characterised in that the lipid has a fatty acyl at the sn-1 and/or sn-2 position of the phosphatidyl group selected from the group consisting of γ-linolenoyl, dihomo-γ-linolenoyl acid and arachidonoyl. Preferences are as for the method above.

Preferred compositions are for treating neurodegenerative conditions, particularly those such as demyelinating diseases, such as multiple sclerosis, Alzheimer's and Parkinsons diseases and the degenerative sequelae associated with head trauma, stroke and intracranial bleeds, whereby neuronal function may be improved or restored from an impaired condition, eg. by remyelination.

Compositions may comprise the pure phospholipid, but it is found that some of these are not stable over periods of weeks and months, at room temperature, in which case they can be stabilised by cold storage or by admixture with a diluent or carrier material. Suitable diluents and carriers can be found in the literature in texts such as Remington's Pharmaceutical Sciences. One particular material investigated by the inventors has been PEG (polyethylene glycol) eg. PEG200. This can be used at amounts between 1 and 99% PEG to 99% to 1% by weight of active phospholipid, preferably 20 to 80% PEG to 80 to 20% of phospholipid and more preferably 40 to 60% PEG to 60 to 40% by weight of phospholipid.

It will be realised by those skilled in the art that other beneficial agents may be combined with the lipids for use in the present invention or otherwise form part of a treatment regime with the lipids. These might be ion channel blockers, eg. sodium channel blockers, interferons (α, β, or γ), T-cell depleters, steroids or other palliative agents. It will further be realised that where the immune and inflammatory responses are being modulated, such combinations will need to be made carefully, given the complex nature of these systems. However, given the delayed response to the present oils, shorter acting agents might be beneficial in the first months of treatment before the TGF-β1 levels are normalised, as long as the additional treatment does not impede this normalization process.

In a fourth aspect of the present invention there are provided novel phospholipids selected from monoacyl or diacyphosphatidyl compounds of general formula 1 containing at least one γ-linolenoyl, dihomo-γ-linolenoyl or arachidonoyl group

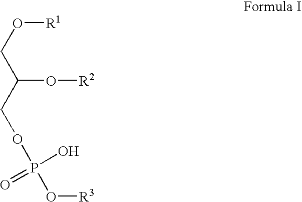

wherein R¹ and R² are independently selected from the group consisting of hydrogen, γ-linolenoyl, dihomo-γ-linolenoyl and arachidonoyl, C₃₋₃₆ mono-unsaturated fatty acyl, linoleoyl or n-3 polyunsaturated acyl groups and optionally substituted C₂₋₃₆ saturated acyl, and

R³ is selected from moieties that are found conjugated to phosphatidyl groups in mammalian cell membranes

with the condition that ONLY one of R¹ and R² MUST be selected from γ-linolenoyl, dihomo-γ-linolenoyl acid and arachidonoyl and the other is not one of these fatty acyl groups.

For the purpose of the present invention the fatty acyl groups R¹ and R² comprise at least one carbonyl group on the end of a hydrocarbyl chain selected from alkyl and alkenyl chains, the carbonyl group being directly attached by its carbon to the oxygen of the glycerol residue shown in Formula 1

Preferred acyl groups R¹ and R², when they are not γ-linolenoyl, dihomo-γ-linolenoyl and arachidonoyl, are saturated acid moieties, preferably fatty acyl, of formula —CO—(CH₂)ₙ—CH₃, wherein n is an integer selected from 1 to 22, more preferably being 4 to 16, still more preferably being from 5 to 12, most preferably being from 6 to 10. Particularly preferred acyl groups are those of caprylic and capric acids, particularly being 1 ,3-dicaprylic or 1 ,3-dicapric glycerols having the γ-linolenic acid, dihomo-γ-linolenic acid or arachidonic acid moiety at the sn-1 or sn-2 position, most preferably the sn-1 position.

Preferred groups R³ include choline, ethanolamine, serine, inositol and glycerol as described fro the method. Other naturally occuring groups R³ will occur to those skilled in the art in the light of these but may be tissue specific, eg. found in T-cells or nervous tissue, eg. neurons.

Similarly, other preferred acids for sn-1 and sn-2 are selected from fatty acids that are metabolised in the human to yield energy as opposed to a fatty acid that is primarily directed to the structural membrane pool: such preferred acids include palmitic acid.

The fatty acyl groups may be optionally substituted and these substitutions preferably are by hydroxy, oxo, carboxyl, alkyl, alkenyl and alkoxy groups.

A sixth aspect of the present invention provides method of synthesis of the novel and known compounds of the invention as set out herein in the schemes below.

Several of the compounds for use in the method of the present invention are known and can be produced by methods as set out in the attached references which are incorporated herein by reference. G indicates γ-linolenic acid, O indicates Oleic acid, A indicates arachidonic acid, DHLA indicates dihomo-γ-linolenic acid, DOCO is docosohexanoic acid, and C is decanoyl (saturated) residue in each case. S is serine and Ln is linoleic and P is palmitoyl.

GGPc ^{1,2}, GOPc ², AAPc ^{3,4}, DHLA(DHLA)Pc ^{5,6}, DOCO(DOCO)Pc ^{4,7}, OOPc ⁸ and CCPc ⁹ are all known compounds. However, GCPc and CGPc are believed novel. Chemie Linz ¹⁰ which describes POPC, SOPC, SLPc, and SAPc. PAPC ¹¹ and SLnPc ¹² have also been prepared.

A preferred exemplary known phospholipid for use in the method, composition and use of the invention 1,2-Di(γ-linolenyl)-sn-glycerophosphocholine GGPc

The invention provides a first method of its preparation of compounds GGPc, DHGLADHGLAPc and AAPc in a one step process (scheme 5, method A) by reaction of sn-glycerophosphocholine cadmium complex with γ-linolenic, dihomo-γ-linolenic, or arachidonic anhydride (formed eg. from the n-6 acid and dicylohexylcarbodiimide). Purification of the crude product uses eg. copper sulfate washes to remove DMAP and column chromatography to take out other by-products. The first batch of product for the γ-linolenic product (44 g, 85% purity) was obtained as a yellow wax. Over 6 months this material was found to have deteriorated to only 70% purity and was now a brown colour suggesting oxidation had occurred.

On a 7 g scale higher purity GGPc could be obtained by more rigorous chromatography. Furthermore freeze-drying a dioxan solution of this material gave a white solid. This solid was hygroscopic and gained weight and went to a foam on prolonged exposure to the atmosphere.

An improved procedure (scheme 5, method B) was used which reacted sn-glycerophosphocholine cadmium complex with γ-linolenic (or other n-6 acid) imidazolide (from eg. GLA and carbonyldiimidazole) in the presence of dimsyl sodium in DMSO. This reaction is much faster and cleaner since there is less contamination by reactant by-products and excess reagents. Purification by chromatography and freeze-drying is still preferable but is much easier in this case. The batch of GGP obtained was a tan sticky solid (93% purity: the starting GLA is 95% pure (Scotia) and two GLA residues are incorporated into the phospholipid). This was stored at −20° C. under nitrogen. The white solid can be obtained by freeze-drying concentrated dioxan solutions.

A process for providing the preferred novel phospholipids of the invention such as GCPc (5) includes a 4 step route shown in scheme 4 which also uses sn-glycerophosphocholine cadmium chloride complex as starting material. The first stage intermediate, 1-trityl-sn-glycerophosphocholine (TritylPc, 2), is known ^{10,13} and was prepared it by carrying out the literature procedure on a larger scale and in a modified manner.

The second step intermediate TritylCPc (3) and the products GCPc, DHGLACPc and ACPc are novel. Synthesis involves the acylation at the 2-position of the tritylglycerophosphocholine (TritylPc, 2). Reaction of the latter with either the saturated fatty acyl chloride or anhydride, eg decanoyl chloride or decanoyl anhydride (from decanoic acid ad DCC) in chlorinated solvents gives low yields of the desired product. When the sodium salt of TritylPc was formed in DMSO by reaction of TritylPc with dimsyl sodium it underwent rapid acylation with decanoyl imidazolide (from decanoic acid and carbonyl diimidazole) to give TritylCPc (3).

The third and fourth steps consist of deprotection of the trityl group of TritylCPc using BF₃etherate followed by acylation at the 1-hydroxyl group of intermediate 4 with γ-linolenoyl anhydride to yield GCPc (5). Use of the anhydride corresponding dihomo or arachidonyl anhydride accesses the corresponding final products.However, initial attempts focused on the ‘two steps in one pot strategy’ which had been reported to work ¹³ to give phosphocholines such as POPe uncontaminated with OPPc (by migration of the 2-acyl group to the 1-position in the deprotected intermediate). When it was tried to apply this methodology to GCPc first was only obtained the deprotected intermediate 4. Eventually a very low yield of GCPc ( 2%.).

To circumvent these problems steps 3 and 4 are preferably carried out separately. Deprotection may be effected by acetic acid at 55° C.; acylation by γ-linolenoyl (or corresponding other n-6 acid) anhydride catalysed by DMAP. These reactions give better yields on a small-scale.

The route that has been provided to prepare CGPC 5 and its corresponding dihomo and arachidoyl compounds is shown in scheme 1. It starts from glycerophosphocholine cadmium chloride complex 1 and uses trityl protected intermediates 2 and 3 to control the regiochemistry of the fatty acid chains at the sn-1 and sn-2 positions. The most problematical step is the deprotection. The key to success lies in the ability to monitor reactions because of the UV-absorbing properties of the GLA unit. Deprotecting agents such as dilute hydrochloric acid in dioxan at 80° C. or formic acid in ether at room temp appear to have worked well.

A second, one-stage more direct route (scheme 2) is provided which involves the sequential acylation of glycerophosphocholine with unsaturated fatty acyl , eg decanoyl, imidazolide and then GLA, dihomo-GLA or arachidonyl-imidazolide. Although mixtures (and hence substantial purification) may be expected the problems associated with protecting groups are avoided.

GCPc (8) (or corresponding dihomo or arachidonyl compound) may be prepared by the 4 step route shown in scheme 3 which also uses sn-glycerophosphocholine cadmium chloride complex 1 as starting material and trityl protected intermediates. The route is similar to that in scheme 1 for CGPC but the fatty acids are introduced in reverse order.

References

1. T. Rezanka and M. Podojil, i J. Chromatogr. A, 463, 397-408 (1989) Preparative separation of algal polar lipids and of individual molecular species by high-performance liquid chromatography and their identificationby gas chromatography-mass spectrometry.

2. D. Horrobin, A. Mc|Mordie and M. S. Manku, Eur. Pat. Appl. EP 609078 A1 3 Aug. 1994 (Scotia Holdings PLC).Phospholipids containing two different unsaturated fatty acids for use in therapy, nutrition and cosmetics.

3. F. H. Chilton and R. C. Murphy, Biophys. & Biochem. Res. Comm., 145, 1126-1133 (1987)

4. C. J. Dekker, W. S. M. Geurts van Kessel, J. P. G. Klomp, J. Pieters and B. De Kruijff, Chem. Phys. Lipids, 33, 93-106 (1983).

5. C. S. Ramesha and W. C. Pickett, J. Lipid Res., 28, 326-331 (1987)

6. E. L. Pugh and M. Kates, J. Biol. Chem., 252, 68-73 (1977)

7. N. Fukuda, H. Nobuo and O. Nakachi, Japanese Patent JP 01141598 A2 2 Jun. 1989 (Nippon Oils & Fats Co.)

8. T. G. Warner and A. A. Benson, J. Lipid Res., 18, 548-551 (1977)

9. A. J. Slotboom, R. Verger, H. M. Verheij, P. H. M. Baartmans, L. L. M. Van Deenen and G. H. De Haas, Chem. Phys. Lipids, 17, 128-147 (1976).

10. F. Paltauf and A. Hermetter, U.S. Pat. No. 4,622,180 11-11-1986 (Chemie Linz A G Derivatives of glycerophosphocholine and glycerophosphoethanolamine, their preparation and their use

11. Jie Xia and Yong-Zheng, Chem. Pharm. Bull., 47, 1659-1663 (1999)

12. Jie Xia and Yong-Zheng, Bioorg. Med. Chem. Lett., 5, 1919-1922, (1995)

13. G. Borsotti, G. Guglielmetti, S. Spera and E. Battistel, Tetrahedron, 57, 10219-10227 (2001).Synthesis of Phosphatidylcholines containing Ricinoleic acid.

The present invention will now be described by way of illustration only by reference to the following non-limiting synthesis and biology Examples, Tables and Figures. Further embodiments falling within the scope of the claims will occur to those skilled in the art in the light of these.

### FIGURES

FIG. 1: Shows the synthetic route for synthesis of CGPc.

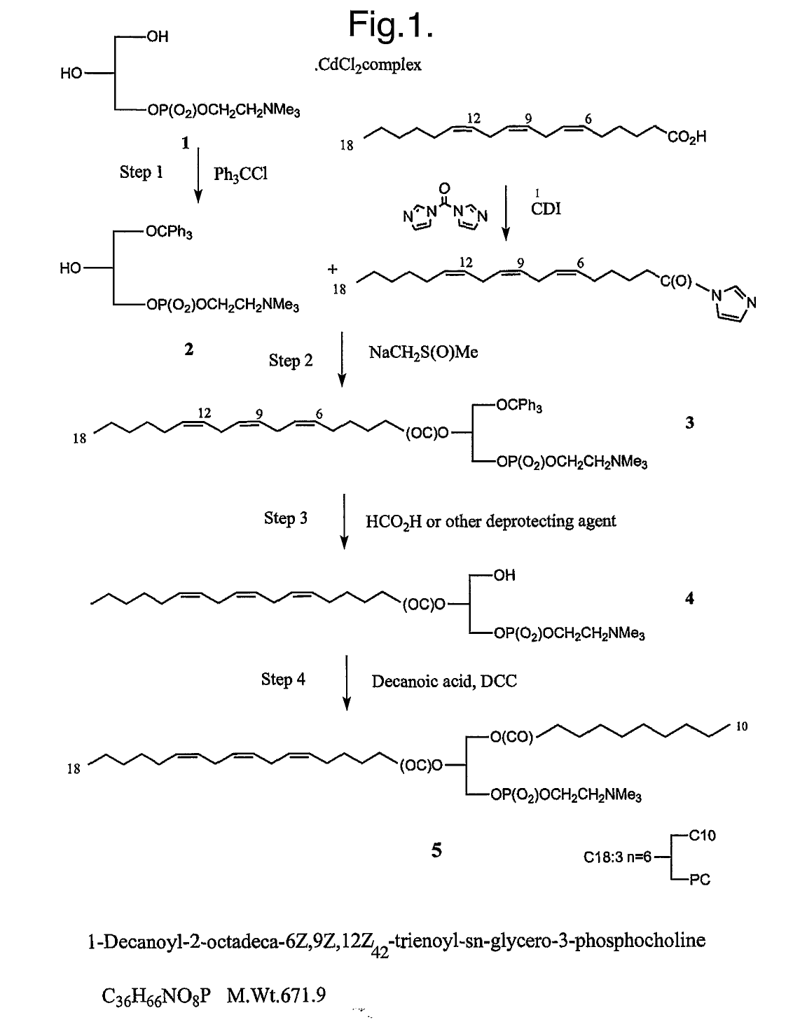

FIG. 2: Shows the synthetic route for synthesis of CGPc.

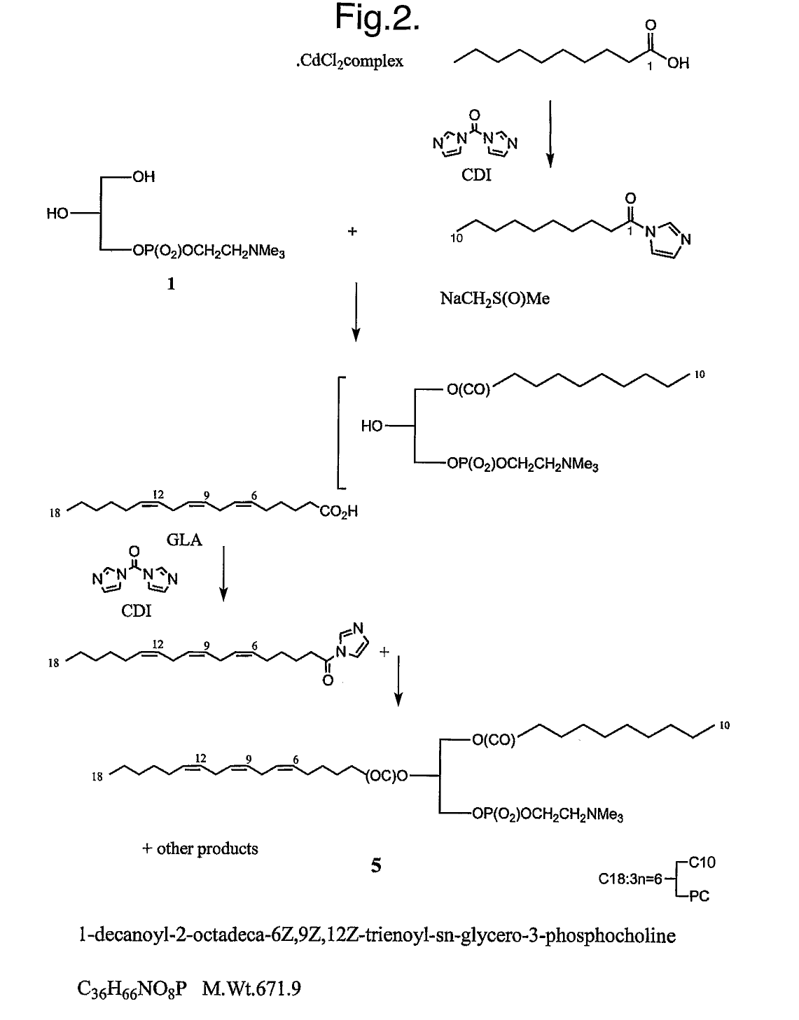

FIG. 3: Shows a synthetic route for synthesis of GCPc.

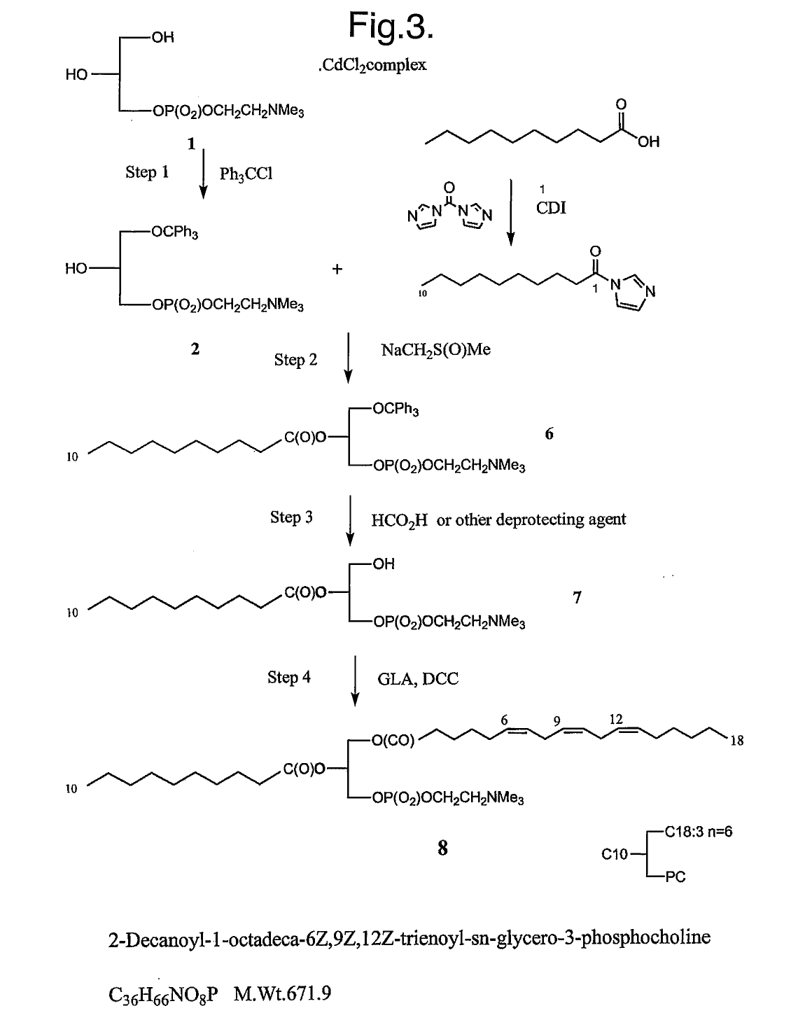

FIG. 4: Shows a synthetic route for synthesis of GCPc.

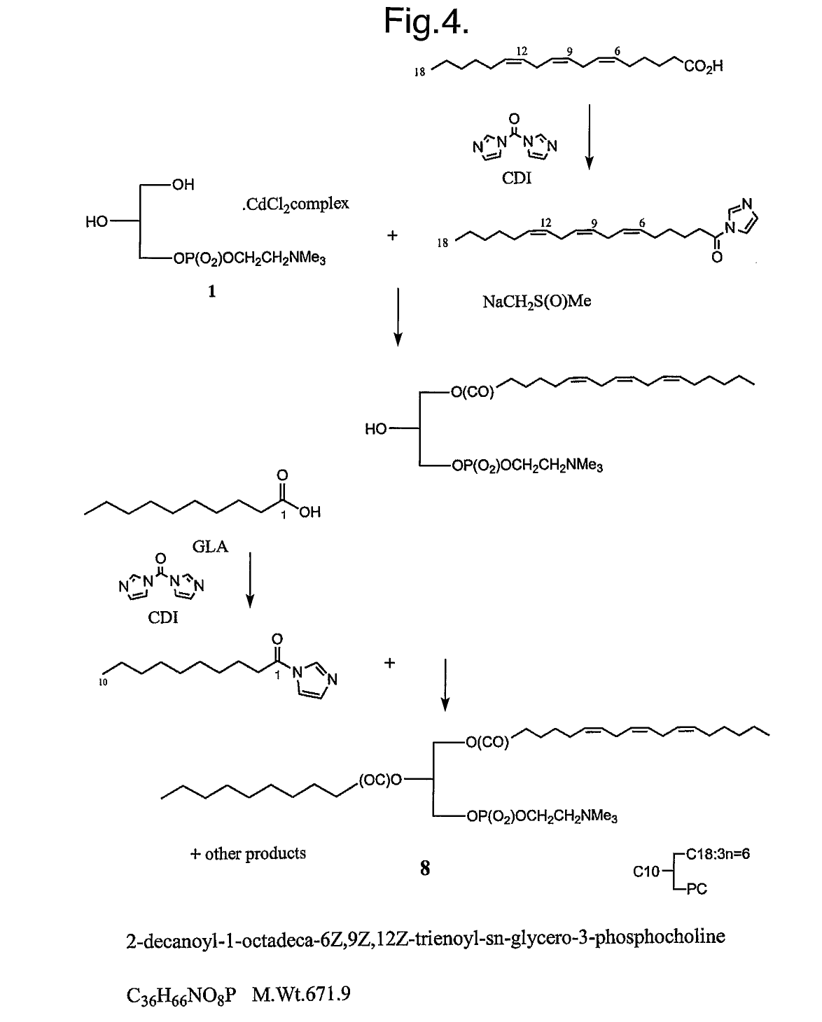

FIG. 5: Shows the synthetic route for synthesis of GGPc.

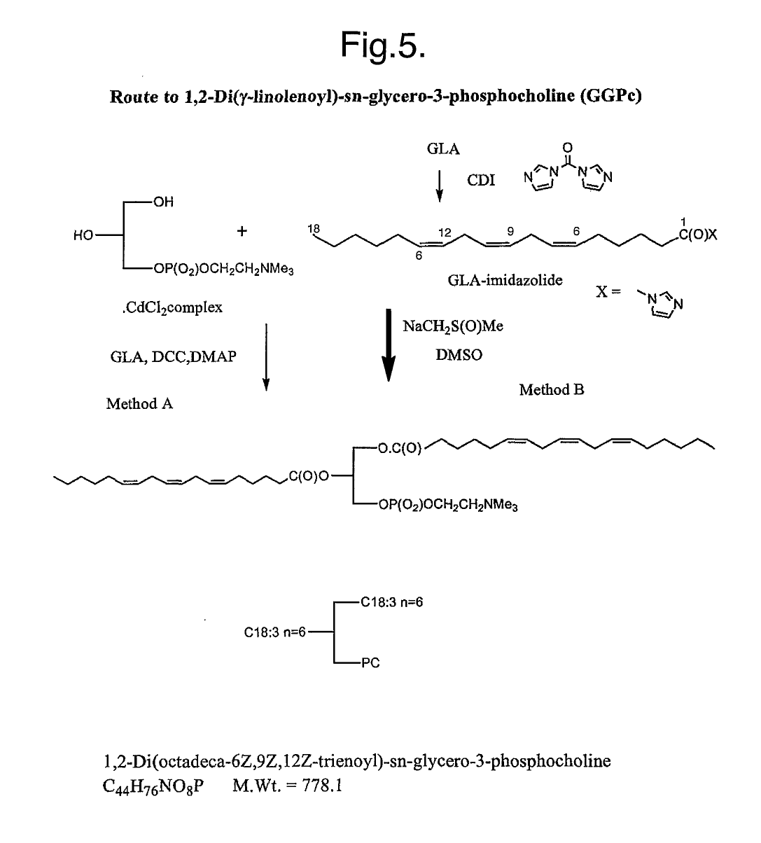

### NMR

Proton-decoupled ¹³C NMR spectra with suppressed NOE were collected at 21° C. in a 5-mm broadband probe on a Joel 500 MHz spectrometer operating at 125.728 MHz. Waltz decoupling was the chosen mode of decoupling and was gated on only during the 14.89 s acquisition time. The relaxation delay was set at 30 secs and the pulse angle was 90°. The spectral window used was ca.35 ppm (from 173.5 to 172.6 ppm) with a 170 ppm offset. The spectra were internally referenced to CDCl₃ at 77.0 ppm. Typically, the approximate number of scans collected for adequate signal-to-noise ranged from 300 to 1200 scans depending on the concentration and purity of the sample. The total acquisition time for the experiments ranged between 2-8h e.g 1272 scans; data points 65,536. Concentrated solutions up to 20% w/v were employed when possible to reduce the acquisition time The chemical shifts quoted vary with the concentration of the solution.

### EXAMPLE 1

### EXAMPLE 1a

### 1,2-Di(γ-linolenyl)-sn-glycerophosphocholine GGPc Method A

To a solution of γ-linolenic acid (138 g, 0.5 mol, 4 equiv) in DCM (750 ml) was added dicyclohexylcarbodiimide (56.4 g, 0.275 mol, 2.5 equiv) and the reaction mixture was stirred under a nitrogen atmosphere for 3 h at room temperature.

To the resulting mixture was added sn-glycerophosphocholine.CdCl2 complex (55 g, 0.124 mol, 1 equiv; previously dried overnight in a vacuum oven at 75° C. over P₂O₅), followed by DMAP (30.3 g, 0.248 mol, 2 equiv). The reaction mixture was stirred for 72 h in the dark at room temperature, then filtered (Celite), the filter pad washed with DCM (200 ml) and the combined filtrate and washings washed with copper sulfate solution (2×300 ml)[ to remove DMAP]. After drying over MgSO₄ the solvent was removed in vacuo. Ethanol and toluene were added to aid complete removal of water. The residual foam was chromatographed on silica (500 g), eluting with DCM-MeOH and gradually increasing the proportion of methanol from 0 → 60%. Combination and concentration of appropriate fractions afforded product (55 g) as a tan wax. HPLC purity 85.1%. δ_{c} (125.7 MHz, CDCl₃) 172.99 (1C, sn-2 carbonyl), 173.21 (1C, sn-1, 3 carbonyls).

### EXAMPLE 1b

### 1,2-Di(γ-linolenyl)-sn-glycerophosphocholine GGPc Method B

1,1′-Carbonyldiimidazole (CDI, 52.0 g, 0.32 mol, 3.2 equiv) was added in portions to a solution of y-linolenic acid (83.4 g, 0.30 mol, 3 equiv) in dry THF (500 ml) and the mixture stirred at room temperature for 1h under nitrogen. The solvent was removed in vacuum. The sn-glycero-3-phosphocholine.CdCl₂ complex (1, 44.0 g, 0.10 mol, 1 equiv; previously dried overnight in a vacuum oven at 75° C. over P₂O₅) was added to the residue and dissolved in dry DMSO (800 ml). A freshly prepared solution of dimsyl sodium [from Na metal (11.5 g, 0.50 ga, 5 equiv) in 500 ml DMSO] was then added at such a rate that the temperature stayed below 50° C. The addition takes ca. 0.5h. The reaction mixture darkened, was stirred for 30 min and then poured onto aqueous acetic acid (3L water-100 ml acetic acid). A sticky solid was collected by filtration and washed with water (1L). This material was dissolved in toluene (1.5 L) the solution washed with water (1L) and both layers filtered through glass fibre filters (to remove cadmium residues). The filtrate was warmed to 70° C. ( to break up the emulsion) , the toluene layer separated and the solvent removed in vacuo to give a dark viscous oil (150 g). This material was chromatographed on silica ( 1 Kg). Elution with CH₂Cl₂-MeOH (30:70) and then methanol gave a tan syrup. The methanol fractions were analysed by HPLC and those of purity >93% combined. The impure fractions were re-chromatographed to give further product.

The above procedure was repeated on the same scale and the products combined. This material was freeze-dried from dioxan (1.5 L) to give a 70 g of a tan sticky solid (HPLC purity 93%).

### EXAMPLE 2

### Experimental (Scheme 1) CGPc 1-Decanoyl-2-γ-linolenoyl-sn-glycero-3-phosphocholine (5)

Stage 1. 1-O-Triphenylmethyl-sn-glycero-3-phosphocholine (2)

sn-Glycero-3-phosphocholine cadmium complex (1, 50.0 g, 0.11 mol, dried in a vacuum oven at 70° C.) and triphenylmethyl chloride (trityl chloride, 46.0 g, 0.16 mol) were dissolved in dry dimethylformamide (400 ml) at 70° C. Triethylamine (23 ml, 19.9 g, 0.19 mol) was then added and the mixture stirred for 0.5h at 70° C. with exclusion of moisture. When the reaction mixture had cooled powdered NaHCO₃ (50 g, 0.6 mol) was added and the mixture stirred for 20 min at room temperature. Inorganic salts were removed by filtration and the filtrate poured onto diethyl ether (1 L) . The mixture was shaken and the ether decanted off. The residue was shaken with ether (2×150 ml), the ether decanted off, and the remaining oily material dissolved in dichloromethane (500 ml). Isobutanol (250 ml) was added, the resulting solution washed with 4% aqueous NH₃ solution and then filtered through celite. The organic layer was washed with water, dried over MgSO₄, and stood overnight at 4° C. A pale yellow solid deposited. This material was removed by filtration, washed with ether and dried in vacuo to give 20.2 g (36%) of the product (99% purity by HPLC)

Stage 2 1-Triphenylmethyl-2-γ-linolenoyl-sn-glycero-3-phosphocholine (3)

1,1′-Carbonyldiimidazole (CDI, 16.4 g, 0.10 mol, 2.2 equiv) was added in portions to a solution of γ-linolenic acid (GLA, 23.3 g, 0.084 mol, 1.9 equiv) in dry THF (325 ml) and the mixture stirred at room temperature for 2h under nitrogen. The solvent was removed in vacuo to give a semi-solid residue. A solution of 1-O-triphenylmethyl-sn-glycero-3-phosphocholine (2, 22.0 g, 0.045 mol, 1 equiv) in dry DMSO (125 ml) was then added to this residue followed by a freshly prepared solution of dimsyl sodium [from Na metal 2.93 g (0.13 ga, 2.8 equiv) in 220 ml DMSO at 60° C. for 0.5h]. The reaction mixture turned to a gel almost immediately, was shaken and stirred for 30 min and then poured onto aqueous acetic acid (1.2 L of 0. 1N). The oily mixture was extracted 2× with dichloromethane-methanol (2:1), the combined extracts washed 2× with methanol-water (1:1) to remove DMSO, and then dried over MgSO₄. Removal of the solvent in vacuo gave a tan oil (43 g. ). This material was chromatographed on silica (500 g). Elution with dichloromethane and then CH₂Cl₂-MeOH-NH₃soln (75:25:2) gave a yellow by-product. Further elution with MeOH-NH₃ soln (98:2) gave the product (19.9 g) as a white waxy solid of low mp (HPLC purity 95.7% )

Stage 3 2-γ-linolenoyl-sn-glycero-3-phosphocholine (4)

Formic acid (60 ml, 1.6 mol) was added to a solution of 1-O-triphenylmethyl-2-γ-linolenoyl sn-glycero-3-phosphocholine (3, 4.55 g, 0.01 mol) in diethyl ether (40 ml). The mixture was stirred at room temperature for 1h under N₂ and then concentrated in vacuo. The residue was dissolved in ether and again concentrated in vacuo to remove more formic acid. The residual syrup was triturated in hexane, and the hexane layer decanted; the process was then repeated with hexane-ether (1:1, 2×) and then ether (2×). The remaining gum was chromatographed on silica. Elution with dichloromethane-methanol mixtures (100:0 to 0:100) and then 2% NH₃ solution in methanol gave 2.2 g of the product as a colourless glass HPLC purity 96.9%

δ_{H} (500 MHz, CDCl₃) 0.89 (3H, t, J=6.7 Hz, C-CH₃), 1.24-1.41(8H, m, 4 × CH₂), 1.61(2H, m, CH₂—C—C═O), 2.06 (4H, m, 2 × CH₂—C═C), 2.32 (2H, t, J=7.5 Hz, CH₂C═O), 2.80 (4H, m, 2 × C═CH₂C═C), 3.33 (9H, s, NMe₃), 3.69 (2H, m, CH₂N), 3.78-3.89 (2H, m, glycerol sn-1 OCH₂), 3.9-4.0 (1H, m, glycerol sn-3 OCHH), 4.08 (1H, m, glycerol sn-3 OCHH), 4.28-4.38 (3H, m, POCH₂+glycerol sn-2 OCH), 5.28 (6H, m, olefinic H).

Stage 4 1-Decanoyl-2-γ-linolenoyl-sn-glycero-3-phosphocholine (5)

Dicyclohexylcarbodiimide (12.7 g, 62 mmol, 10.5 equiv ) was added to a solution of decanoic acid (20.2 g, 0.12 mol, 20 equiv) in dry dichloromethane (80 ml) under N₂. The mixture was stirred for lh and the precipitated dicylcohexylurea (DCU) removed by filtration. The filtrate, a solution of decanoic anhydride in dichloromethane, was added to a stirred solution of 2-γ-linolenoyl-sn-glycero-3-phosphocholine (4, 3.04 g, 5.9 mmol, 1 equiv.) in dichloromethane (40 ml). 4-Dimethylaminopyridine (0.72 g, 5.9 mmol) was then added and the mixture stirred for 3h. The mixture was filtered to remove DCU and the filtrate concentrated in vacuo. The oily residue (25 g) was chromatographed on silica. Elution with dichloromethane -methanol mixtures (100:0 to 0:100) and then methanol -ammonia solution (98:2) gave 2.0 g of the product as a colourless glass (HPLC purity 92.1%). A sample was freeze-dried from dioxan to give a waxy solid (HPLC purity 97.5%).

δ_{H} (500 MHz, CDCl₃) 0.89 (6H, 2× t, J=6.7 Hz, 2× C-CH₃), 1.24-1.43 (20H, m, 10× CH₂), 153-1.65(4H, m, 2× CH₂—C—C═O), 2.07 (4H, m, 2× CH2—C═C), 2.30 (2H, m, 2× CH₂C═O), 2.80 (4H, m, 2× C═CH₂C═C), 3.35 (9H, s, NMe₃), 3.82 (2H, m, CH₂N), 3.95 (2H, m, glycerol sn-3 OCH₂), 4.12 1H, m, sn-l OCHH), 4.3-4.5 (3H, m, POCH₂+glycerol sn-1 OCHH), 5.20 (1H, m, glycerol sn-2 OCH), 5.26-5.44 (6H, m, olefinic H).

### EXAMPLE 4

Scheme 2

GCPc 1-γ-linolenoyl-2-decanoyl-sn-glycero-3-phosphocholine (8)

Stage 1. 1-O-Triphenylmethyl-sn-glycero-3-phosphocholine (2) as above

Stage 2 1-Triphenylmethyl-2-decanoyl-sn-glycero-3-phosphocholine (6)

1,1′-Carbonyldiimidazole (CDI, 13.0 g, 0.080 mol, 2.3 equiv) was added in portions to a solution of decanoic acid (11.5 g, 0.0067 mol, 1.9 equiv) in dry THF (250 ml) and the mixture stirred at room temperature for 2h under nitrogen. The solvent was removed in vacuo to give a semi-solid residue. A solution of 1-O-triphenylmethyl-sn-glycero-3-phosphocholine (2, 17.5 g, 0.035 mol, lequiv) in dry DMSO (85 ml) was then added to this residue followed by a freshly prepared solution of dimsyl sodium [from Na metal 2.36 g (0.12 ga, 3 equiv) in 175 ml DMSO at 60° C. for 0.5h]. The reaction mixture turned to a gel almost immediately, was shaken and stirred for 30 min and then poured onto aqueous acetic acid (1 L of 0.1N). The oily mixture was extracted 2× with dichloromethane-methanol (2:1), the combined extracts washed 2× with methanol-water (1:1) to remove DMSO, and then dried over MgSO₄ . Removal of the solvent in vacuo gave a viscous oil which was dissolved in ether (50 ml). The ether solution was washed with 2×5% aqueous NaCl, treated with decolourising charcoal and dried over MgSO₄. The solvent was removed in vacuo, stirred with dichloromethane (30 ml) for 5 min, the insoluble material (no UV absorption) removed by filtration and the filtrate concentrated in vacuo to a gum (19 g). This material was chromatographed on silica (250 g). Elution with dichloromethane and then CH₂Cl₂-MeOH-NH₃soln (70:30:2) gave a yellow by-product. Further elution with CH₂Cl₂-MeOH-NH₃soln (50:50:2) gave the product (14.1 g) as a white solid of low mp (HPLC purity 98% ) δ_{H} (500 MHz, CDCl₃) 0.93 (3H, t, J=7.0 Hz, C—CH₃), 1.29 (12H, m, 6× CH₂), 1.67(2H, m, CH₂—C—C═O), 2.40 (3H, t, J=7.5 Hz, CH₂C═O), 3.22 (9H, s, NMe₃), 3.29 (2H, m, CH₂N), 3.63 (2H,m, OCH₂), 3.99 (2H, m, OCH₂), 4.17 (4H, m, OCH₂+H₂O), 5.29 (1H, m, OCH), 7.30 and 7.45 (15H, m, aromatics).

Stage 3 2-Decanoyl-sn-glycero-3-phosphocholine (7)

Formic acid (160 ml, 1.6 mol) was added to a solution of 1-O-triphenylmethyl-2-decanoyl-sn-glycero-3-phosphocholine (6, 8.5 g, 0.01 mol) in diethyl ether (80 ml). The mixture was stirred at room temperature for 1h under N₂ and then concentrated in vacuo. The residue was dissolved in ether and again concentrated in vacuo to remove more formic acid. The residual syrup was triturated in hexane, and the hexane layer decanted; the process was then repeated with hexane-ether (1:1, 2×) and then with ether (4×). The remaining gum was freeze-dried from dioxan to give 4.2 g of the product as a waxy solid.

Stage 4 2-Decanoyl-1-γ-linolenoyl-sn-glycero-3-phosphocholine (8)

Dicyclohexylcarbodiimide (12.7 g, 62 mmol, 10.5 equiv ) was added to a solution of γ-linolenic acid (GLA, 33.4 g, 0.12 mol, 20 equiv) in dry dichloromethane (80 ml) under N₂. The mixture was stirred for 1h and the precipitated dicylcohexylurea (DCU) removed by filtration. The filtrate, a solution of decanoic anhydride in dichloromethane, was added to a stirred solution of 2-γ-linolenoyl-sn-glycero-3-phosphocholine (7, 3.04 g, 5.9 mmol, 1 equiv.) in dichloromethane (40 ml). 4-Dimethylaminopyridine (0.72 g, 5.9 mmol, 1 equiv) was then added and the mixture stirred for 3h. The mixture was filtered to remove DCU and the filtrate concentrated in vacuo. The oily residue (25 g) was chromatographed on silica. Elution with dichloromethane -methanol mixtures (100:0 to 0:100) and then methanol-ammonia solution (98:2) gave 2.0 g of the product as a colourless glass (HPLC purity 92.1%). A sample was freeze-dried from dioxan to give a waxy solid (HPLC purity 96.4%).

Biological Studies.

Chronic Relapsing Experimental Autoimmune Encephalomyelitis (CREAE) Studies.

Induction and Clinical Assessment of EAE

CREAE was induced in C57B1/6 and SJL mice. Animals were injected subcutaneously with 100 μg of the neuroantigen peptide MOG 35-55 (amino acid sequence MEVGWYRSPFSRVVHLYRNGK Genemed Synthesis, Inc) or 1 mg of mouse spinal cord homogenate (SCH), in phosphate buffered saline (PBS), emulsified by sonication for 10 min at room temperature, in incomplete Freund's adjuvant (DIFCO, Detroit, USA) supplemented with 480 μg of mycobacteria tuberculosis and 60 μg of Mycobacteria butyricium (DIFCO, Detroit, USA) on days 0 and 7 as described previously (Morris-Downes, M M., et al 2002). In addition to optimise the disease mice also received 200 ng (intraperitoneally) of Bordetella pertussis toxin dissolved in PBS administered 1hr and 24 hrs after immunization with the MOG neuroantigen and for SCH days 0, 1, 7 and 8

Animals were weighed from day 5 onwards and examined daily for clinical neurological signs by two experienced investigators and graded according to a previously validated grading scheme (Morris-Downes, M M. et al 2002 and others): 0=normal; 1=limp tail and feet; 2=impaired righting reflex; 3=partial hind limb paralysis; 4=complete hindlimb paralysis; 5=moribund; 6=death. Animals exhibiting clinical signs of a lesser severity grade than typically observed were scored as 0.5 less than the indicated grade.

Reference

Morris-Downes, M M., et al (2002). Pathological and regulatory effects of anti-myelin antibodies in experimental allergic encephalomyelitis in mice. J. Neuroimmunol. 125. 114-124.

The mean group EAE score was compared for each test group compared to a respective control group by non-parametric statistical analysis (Mann Whitney U Test).

All MOG-CREAE studies comprised a treatment control group (saline). Each structured phospholipid was tested at 3 dose levels, all treaments being orally administered for 2 weeks from day 7 after inoculation. All treatment groups will contained 10 animals. On completion of studies (day 21), brain and spinal cord were be removed and half of the samples were processed for signs of CNS perivascular mononuclear leucocyte-infiltrated sites and demyelination.

Results

Initial results confirm that GGPc was superior in efficacy in the CREAE model MOG in C57BL mice to the lipid GGG of PCT/GB2004/003524. GGPc shows excellent protection at 100 ul. It was tested at 4 doses (25, 50, 100 and 200 microL) against CCC (150 microL) and PEG (the diluant for GGPC) and as part of a very large study which included CGC (0134) and 884.

The dose response curve showed a significant effect (p<0.050 compared to CCC) at the 100 microL dose, but no effect at a higher dose of 200microL. The control EAE showed the disease was modelled well (max score of 3.5-4) and CGC (prior compounds of the inventors) was effective at 100 microL. This compared to 50 microL in previous studies where the disease state was less severe.

GGPc also showed bell shaped curve and provided protection/reduced severity at 100 ul of 50% phospholipid in PEG200 By comparison high sn-2 Borage oil showed effect at 350ul.

Measurement of PBMC cytokines

Isolation and Culture of PBMC

Heparinised whole blood was diluted with an equal volume of Hanks' balanced salt solution (Sigma, UK) and the resulting diluted blood layered onto Lymphoprep (Nycomed, Oslo, Norway). Following density centrifugation at 800 g for 30 minutes the PBMC were removed from the interface and diluted in Hanks' solution. The cells were then washed twice by centrifugation for 10 minutes at 250 g. The resulting final pellet was then resuspended in culture medium consisting of RPMI-1640 medium (Sigma, UK) supplemented with 2 mM L-glutamine, 100U penicillin and 100 μg streptomycin (Sigma, UK) and 10% autologous plasma. 2×10⁶ per ml PBMC, >95% viable as judged by trypan blue exclusion, were added to tissue culture tubes (Bibby Sterilin Ltd, Stone, UK) and incubated for 24h at 37° C. with 5% CO₂. The concentration of antigen, cell density and time of culture were all determined in previous kinetic experiments to determine maximum cytokine production (data not shown). Routine cytospin preparations were also prepared for subsequent differential counts. Following incubation the cells were removed from culture by centrifugation at 250 g for 10 minutes, the resulting supernatants were then removed, aliquoted and stored at −70° C.

Preparation of Plasma Samples

10 ml of heparinised blood was spun at 250 g for 10 minutes. The resulting plasma layer was then removed, aliquoted and stored at −70° C.

Detection of Pro-inflammatory Cytokines

TNF-α, IL-1β and IFN-γ in cell culture supernatants and plasma were detected using commercially available paired antibodies enabling cytokine detection in an ELISA format (R&D systems Ltd, Abingdon, UK). The sensitivities for the TNF-α and IFN-γ ELISAs were 15.6-1000 pg/ml and 3.9-250 pg/ml for IL-1β.

Detection of Biologically Active TGF-β1

Biologically active TGF-β1 in cell culture supernatants and plasma were detected using the commercially available Eₘₐₓ ELISA system with a sensitivity of 15.6-1000 pg/ml (Promega, Southampton, UK).

Statistical Analysis

Differences in cytokine production were compared using Student's t-test and Mann-Whitney U-test and were considered significant when p values were less than 0.05.

1. A method of treating a patient in need of therapy for a disease in which cyokines have become dysregulated, or are otherwise capable of modulation to provide therapeutic benefit, comprising administering to that patient a therapeutically effective dose of a phospholipid comprising a phosphatidyl group esterifed with one or more fatty acyl groups, characterised in that the phospholipid has at least one fatty acyl group at the sn-1 and/or sn-2 position of the phosphatidyl group, the fatty acyl group being selected from the group consisting of γ-linolenoyl, dihomo-γ-linolenoyl acid and arachidonoyl.

2. A method as claimed in claim 1 wherein the patient is in need of modulation of transforming growth factor β (TGF-β), particularly TGF-β1.

3. A method as claimed in claim 1 wherein the patient is in need of modulaton of a cytokines selected from TNF-α and IL-1β.

4. A method as claimed in claim 1 wherein the patient is in need of therapy to maintain and/or restore cytokine balance where imbalance is found in diseases of the immune system and in neurodegeneration.

5. A method as claimed in claim 1 wherein the disease is an autommune disease or multiple sclerosis.

6. A method as claimed in claim 1 wherein the phospholipid has a fatty acyl group selected from the group consisting of γ-linolenoyl acid, dihomo-γ-linolenoyl acid and arachidonoyl at only one of the sn-1 or sn-2 positions of the phosphatidyl group, the other position being free hydroxyl or esterifled with a C2 to C36 unsaturated, monounsaturated or polyunsaturated fatty acyl group.

7. A method as claimed in claim 1 wherein the phospholipid has an sn-1 position fatty acyl group selected from γ-linolenoyl, dihomo-γ-linolenoyl and arachidonoyl and an sn-2 position fatty acyl group selected from C2 to C36 unsaturated, monounsaturated or polyunsaturated fatty acyl other than n-6 acids.

8. A method as claimed in claim 6 wherein the other fatty acid is such that it is used in the metabolic pool, eg. being unsaturated or a metabolically acceptable acid such as oleic or palmitic acid.

9. A method as claimed in claim 1 wherein the phospholipid phosphatidyl group is selected from those found in mammalian, particularly human, cell membranes.

10. A method as claimed in claim 9 wherein the phosphatidyl group is selected from those consisting of phosphatidyl-choline, phosphatidyl-ethanolamine, phosphatidyl-serine, phosphatidyl- inositol, and plasmalogens of the above e.g. lyso-phosphatidyl-choline, lyso-phosphatidyl-ethanolamnine, lyso-phosphatidyl-inositol and lyso-phosphatidyl-inositol.

11. A method as claimed in claim 1 wherein the patient is in need of treatment for a neurodegenerative disease involving demyelination.

12. A method as claimed in claim 11 wherein the demyelination is arrested or reversed.

13. A method as claimed in claim 11 wherein the treatment arrests underlying neurodegeneration and restores neuronal function.

14. A method as claimed in claim 11 wherein the method normalises membrane composition, in immune cells and/or neurones, and restores healthy PBMC spontaneuosly released TGF-β1/TNFα ratios and the ratios of TGF-β1 with other PBMC released cytokines.

15. A method as claimed in claim 1 wherein the treatment arrests neurodegeneration in multiple sclerosis.

16. A method as claimed in claim 15 wherein the treatment is for a disease selected from the group consisting of relapsing remitting, primary and secondary progressive and other chronic progressive MS and the restoration, in part or completely, of neuronal function such as measured, eg. By MRI or CAT scan or by EDSS score.

17. A method as claimed in claim 16 wherein the EDSS score preferably is improved by at least one point, more preferably at least 1.5 points and most preferably by at last 2 points over 18 months of daily treatment.

18. A method as claimed in claim 1 wherein the treatment is selected from the group of those for cerebral impairment after stroke, head trauma and intracranial bleeding where there is demyelination or neuronal damage.

19. A method as claimed in claim 1 wherein the treatment is for chronic demyelination such as in Alzheimer's and Parkinson's disease.

20. A method as claimed in claim 1 wherein the phospholipid is administered for a duration and at a dose sufficient to maintain or elevate TGF-β1 levels in the patient to therapeutic levels.

21. A method as claimed in claim 20 wherein the therapeutic levels is that at least consistent with healthy subjects.

22. A method as claimed in claim 20 wherein the dose is such as to produce a TGF-β1/TNF-α ratio spontaneously released from peripheral blood mononuclear cells (PBMCs) isolated from blood of a patient, after 18 months of daily dosing, of 0.4 to 3.0.

23. A method as claimed in claim 21 wherein the level of TGF-β1 spontaneously released from the PBMCs is at least 80 pg/ml 2×10⁶ cells.

24. A method as claimed in claim 23 wherein the level of TGF-β1 released is above 100 pg/ml and most preferably above 140 pg/ml, still more preferably greater than 180 pg/ml.

25. A method as claimed in claim 1 wherein the amount of phospholipid administered daily is between 1 and 30 grams, orally dosed.

26. A method as claimed in claim 1 wherein the phospholipid is a monoacyl or diacylphosphoglyceride, containing the at least one sn-1 or sn-2 γ-linolenoyl, dihomo-γ-linolenoyl or arachidonoyl group, and is of general Formula I 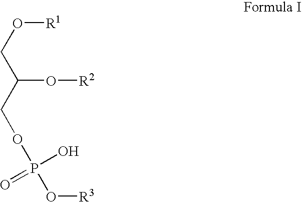
wherein R¹ and R² are independently selected from the group consisting of hydrogen, γ-linolenoyl, dihomo-γ-linolenoyl and arachidonoyl, mono-unsaturated acyl, linoleoyl or n-3 polyunsaturated acyl groups and optionally substituted C₂₋₃₆ saturated acyl, and
R³ is selected from moieties that are conjugated to phosphatidyl groups naturally occurring in mammalian membranes with the condition that one of R¹ and R² must be selected from γ-linolenoyl, dihomo-γ-linolenoyl and arachidonoyl.

27. A method as claimed in claim 26 wherein the acyl groups R¹ and R², when they are not γ-linolenoyl, dihomo-γ-linolenoyl and arachidonoyl, are saturated fatty acyl of formula —CO—(CH₂)ₙ—CH₃, wherein n is an integer selected from 1 to 22, more preferably being 4 to 16, still more preferably being from 5 to 12, most preferably being from 6 to 10.

28. A method as claimed in claim 27 wherein the acyl groups are those of caprylic and capric acids, particularly being 1,3-dicaprylic or 1,3-dicapric glycerols.

29. A method as claimed in claim 26 wherein the group R³ is a polar group selected from the group consisting of choline, ethanolamine, serine, inositol and glycerol.

30. A method as claimed in claim 26 wherein the group R³ is a bipolar group selected from substituted C₁₋₁₀ alkyl or alkenyl groups substituted with eg amine, hydroxy or thio at one end and hydroxyl at the other such that there is formed a phosphotidate ester with the polar group.

31. A method as claimed in claim 6 wherein the non-obligate sn-1 and sn-2 fatty acyl chain (R¹ and R²) is unsaturated and may also be that of other essential fatty acids, such as the n-3 acids such as stearidonic acid, eicosapentanoic acid and docosahexanoic acid.

32. A method as claimed in claim 6 is optionally substituted by hydroxy, oxo, carboxyl, alkyl, alkenyl and alkoxy groups.

33. Use of a compound of formula I, as defined in claim 1 for the manufacture of a medicament for the treatment of disease in which cyokines have become dysregulated, or are otherwise capable of modulation to provide therapeutic benefit.

34. A pharmaceutical composition for the treatment of a patient in need for modulation of dysregulated cytokines or cytokines which are otherwise capable of modulation to provide therapeutic benefit characterised in that it comprises a phospholipid comprising a phosphatidyl group esterifed with one or more fatty acyl groups, characterised in that the lipid has a fatty acyl at the sn-1 and/or sn-2 position of the phosphatidyl group selected from the group consisting of γ-linolenoyl, dihomo-γ-linolenoyl acid and arachidonoyl.

35. A compositions as claimed in claim 34 for treating neurodegenerative conditions, particularly those such as demyelinating diseases, such as multiple sclerosis, Alzheimer's and Parkinsons diseases and the degenerative sequelae associated with head trauma, stroke and intracranial bleeds, whereby neuronal function may be improved or restored from an impaired condition, eg. by remyelination.

36. A composition as claimed in claim 34 comprising the pure phospholipids.

37. A composition as claimed in claim 34 wherein the phospholipid is mixed or otherwise together with a diluent or carrier material.

38. A composition as claimed in any one of claims 34 to claim 34 wherein the diluent or carrier is a polymer.

39. A composition as claimed in claim 38 wherein the diluent or carrier is polyethylene glycol.

40. A composition as claimed in claim 39 wherein the diluent or carrier is PEG200.

41. A composition as claimed in claim 34 wherein the diluent or carrier is present at amounts between 1 and 99% diluent or carrier to 99% to 1% by weight of phospholipid.

42. A composition as claimed in claim 41 wherein the diluent or carrier is present at between 20 to 80% diluent or carrier to 80 to 20% of phospholipid and more preferably 40 to 60% diluent or carrier to 60 to 40% by weight of phospholipid.

43. A composition as claimed in claim 34 further comprising a further therapeutic agent for said disease.

44. A composition as claimed in claim 43 wherein the further therapeutic agent is selected from an ion channel blockers, an interferons (α, β, or γ), a T-cell depleter and a steroid.

45. A phospholipid selected from monoacyl or diacyphosphatidyl compounds of general formula 1 containing at least one γ-linolenoyl, dihomo-γ-linolenoyl or arachidonoyl group 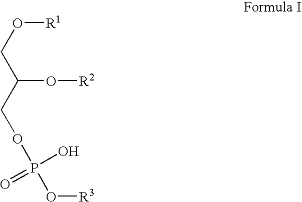
wherein R¹ and R² are independently selected from the group consisting of hydrogen, γ-linolenoyl, dihomo-γ-linolenoyl and arachidonoyl, mono-unsaturated fatty acyl, linoleoyl, n-3 polyunsaturated acyl groups and optionally substituted C₂₋₃₆ saturated acyl, and
R³ is selected from moieties that are found conjugated to phosphatidyl groups in mammalian cell membranes with the condition that ONLY one of R¹ and R² MUST be selected from γ-linolenoyl, dihomo-γ-linolenoyl acid and arachidonoyl and the other is not one of these fatty acid residues.

46. A phospholipid as claimed in claim 45 wherein the acyl groups R¹ and R², when they are not γ-linolenic acid, dilmomo-γ-linolenic acid and arachidonic acid, are saturated acid moieties, preferably fatty acids, of formula —CO—(CH₂)ₙ—CH₃, wherein n is an integer selected from 1 to 22, more preferably being 4 to 16, still more preferably being from 5 to 12, most preferably being from 6 to 10. Particularly preferred acyl groups are those of caprylic and capric acids, particularly being 1,3-dicaprylic or 1,3-dicapric glycerols having the γ-linolenic acid, dihomo-γ-linolenic acid or arachidonic acid moiety at the sn-1 or sn-2 position, most preferably the sn-1 position.

47. A phospholipid as claimed in claim 45 wherein the group R³ is selected from choline, ethanolamine, seine, inositol and glycerol.

48. A phospholipid as claimed in claim 45 wherein the non-obligate sn-1 and sn-2 is selected from fatty acids that are metabolised in the human to yield energy as opposed to a fatty acid that is primarily directed to the structural membrane pool.

49. A method of synthesis A preferred exemplary known phospholipid for use in the method, composition and use of the invention 1,2-Di(y-linolenyl)-sn-glycerophosphocholine GGPc.

50. A method of preparation of compounds selected from the group consisting of (GGPc, DHGLA-DHGLA-Pc and AAPc in a one step process comprising reaction of sn-glycerophosphocholine cadmium complex with γ-linolenic, dihomo-γ-linolenic, or arachidonic anhydride.

51. A method as claimed in claim 50 comprising freeze-drying a dioxan solution of the crude product gave a white solid.

52. A method of preparation of compounds selected from the group consisting of GGPc, DHGLA-DHGLA-Pc and AAPc comprising reacting sn-glycerophosphocholine cadmium complex with a fatty acid selected from the group consisting of γ-linolenic imidazolide, dihomo-γ-linolenic imidazolide and arachidonoyl imidazolide in the presence of dimsyl sodium in polar solvent.

53. A method of preparation as claimed in claim 52 wherein the polar solvent is DMSO.

54. A method of preparation of compounds selected from the group consisting of GGPc, DHGLA-DHGLA-Pc and AAPc comprising reacting 1-trityl-sn-glycerophosphocholine with one of γ-linolenic anhydride, γ-linolenic chloride, dihomo-γ-linolenic anhydride, dihomo-γ-linolenic chloride and arachidonoyl anhydride chloride or arachidonyl anhydride.

55. A method as claimed in claim 54 wherein the sodium salt of TritylPc is acylated with decanoyl imidazolide, the trityl group removed and residue acylated at the 1-hydroxyl group with the n-6 anhydride or chloride.
